(19) United States
(12) Patent Application Publication
(10) Pub. No.: US 2015/0112397 A1
(43) Pub. Date: Apr. 23, 2015

(54) SURGICAL INSTRUMENT AND A SURGICAL SYSTEM FOR FASTENING VERTEBRAE

(71) Applicant: SAFE ORTHOPAEDICS, Eragny Sur Oise (FR)

(72) Inventor: Dominique Petit, Verton (FR)

(73) Assignee: SAFE ORTHOPAEDICS, Eragny Sur Oise (FR)

(21) Appl. No.: 14/389,837

(22) PCT Filed: Apr. 5, 2013
(86) PCT No.: PCT/FR2013/050753

§ 371 Date: Dec. 8, 2014

(30) Foreign Application Priority Data
Apr. 5, 2012 (FR) 1253132

(51) Int. Cl. A61B 17/70

(52) U.S. Cl. CPC A61B 17/7082
USPC 606/86 A

(57) ABSTRACT

A surgical instrument for fastening vertebrae via the posterior approach or via the posterolateral approach, the instrument comprising a tubular element formed of two legs separated by a recessed longitudinal zone, the legs being interconnected at their distal ends. Each of the legs having, in its inside surface, a guide channel of dovetail cross-section and an insert having two diametrically opposite longitudinal slideways of dovetail cross-section complementary to the cross-section of said guide channels. The instrument comprising the proximal end of each of the legs has anchor means suitable for coming to engage in complementary means by a transverse movement of the legs.

### BACKGROUND

The present invention relates to the field of surgical instruments for spinal stabilization operations using bone anchor elements of the screw type via the posterior approach or via the posterolateral approach.

The invention relates more particularly to an instrument kit that is designed particularly, but not exclusively, for lumbar, thoracic, or indeed posterior cervical spinal osteosynthesis surgery via minimally invasive surgery or open surgery.

When anatomical dysfunctions occur in the vertebral column, bone anchors of the vertebral or pedicle screw type are put in place in the vertebrae and connected together by link elements of the rod or plate type.

The Applicant's Patent Application PCT/FR10/000880 is known from the state of the art. That document divulges instrumentation for fastening at least two vertebrae by means of bone anchor implants of the pedicle screw type, that instrumentation comprising: a bone anchor element designed to be fastened to a vertebra, which element is pre-fitted to a single-use fitting tube; and a sterile sealed packaging pack.

That prior art document also relates to an instrument kit for fitting or removing a spinal implant, that kit comprising at least two threaded bone anchor elements, a link member of the rod or plate type for mechanically coupling together the bone anchor elements, and locking elements for locking the link element relative to the anchor elements, for the purpose of performing all of the surgical gestures related to fitting or removing said implant, that instrument kit being characterized in that all of those necessary instruments are single-use instruments, packaged sterile in one or more sealed packs.

Those prior art solutions make it possible to simplify the act of surgery by enabling the surgeon to insert the screw, then to push an intervertebral link rod, and then to put a locking cap in place using a single instrument that remains coupled to the screw throughout that succession of steps.

However, such a prior instrument is sometimes withdrawn before the operation is finalized, e.g. to enable the operation zone to be seen more clearly.

The procedure for re-engaging the instrument onto the head of the pedicle screw is then tedious.

With the prior art instrument, such re-engagement is achieved by tilting the two half-tubes at a large angle, requiring a large incision to accommodate that movement.

The distal end is positioned with the operator having little visibility, and the accuracy of the gesture is thus highly random.

### SUMMARY

An object of the present invention is to remedy that drawback by proposing a novel surgical instrument that is inter-operable with the above-mentioned instrumentation.

In its most general acceptation, the invention provides a surgical instrument for fastening vertebrae via the posterior approach or via the posterolateral approach, the instrument being constituted by:
a tubular element formed of two legs separated by a recessed longitudinal zone and joined at their distal ends by interconnection means;
each of said legs having, in its inside surface, a guide channel of dovetail cross-section; and
an insert having two diametrically opposite longitudinal slideways of dovetail cross-section complementary to the cross-section of said guide channels;
said instrument being characterized in that the proximal end of each of the legs has anchor means suitable for coming to engage in complementary means by a transverse movement of said legs. This movement takes place in a diametrical direction perpendicular to the transverse middle axis passing between the two legs. In the spaced-apart position, the legs can engage onto the head of the screw by a longitudinal axial movement. In the closed position, the fastening means prevents the legs and the head of the screw from moving relative to one another.

It is thus possible to engage the instrument onto the screw as anchored in the pedicle by an axial insertion movement, and then to close the legs, by mechanical action, e.g. by sliding a ring surrounding the legs, by inserting the insert that brings the two legs closer together, or by them returning resiliently into the rest position.

Preferably, the proximal end of each of the legs is provided with a transverse lug suitable for being engaged in a groove in the head of a pedicle screw, by lateral elastic deformation of the legs.

In a preferred embodiment, the maximum height of said anchor means is smaller than the opening in said additional means.

Advantageously, said lug is provided with a distal shoulder extending in a transverse plane perpendicular to the longitudinal axis of the insert.

Preferably, said lug has a distal surface that is substantially conical, for forming an insertion canal that opens out in the proximal direction, said canal having, in the locked position, a shape complementary to the outside envelope of the head of the screw.

In a particular variant, at rest, said legs diverge in the proximal direction.

Advantageously, the legs are mounted to move between a position in which the proximal ends are spaced apart from each other beyond a rest position of said legs, and a position in which the proximal ends are brought closer together by the insert being engaged in the longitudinal zone. In a particular configuration, the insert brings the proximal ends closer together beyond the rest position of the legs.

In a preferred variant, said legs are of semi-cylindrical shape.

In a particular embodiment, the interconnection means for interconnecting the two legs are constituted by an annular distal portion extending said legs.

In another variant, said interconnection means for interconnecting the two legs are constituted by a handle that interconnects said legs.

The invention also provides a surgical instrument system for fastening vertebrae via the posterior approach or via the posterolateral approach, said system being constituted by a first instrument as defined above, and by a second instrument constituted by a tubular element formed of two legs separated by a recessed longitudinal zone and joined at their distal ends by interconnection means, each of said legs having, in its inside surface, a guide groove of dovetail cross-section, the system further being characterized in that said insert is common to the first instrument and to the second instrument.

### BRIEF DESCRIPTION OF THE DRAWINGS

Other objects and advantages of the invention appear from the following description given with reference to the accompanying drawings, in which:

FIG. 1 is a view of the component elements of an instrument of the invention;

FIG. 2 is a detail view in section of the ends of the legs of the surgical instrument of the invention; and

FIG. 3 is a view of a variant embodiment.

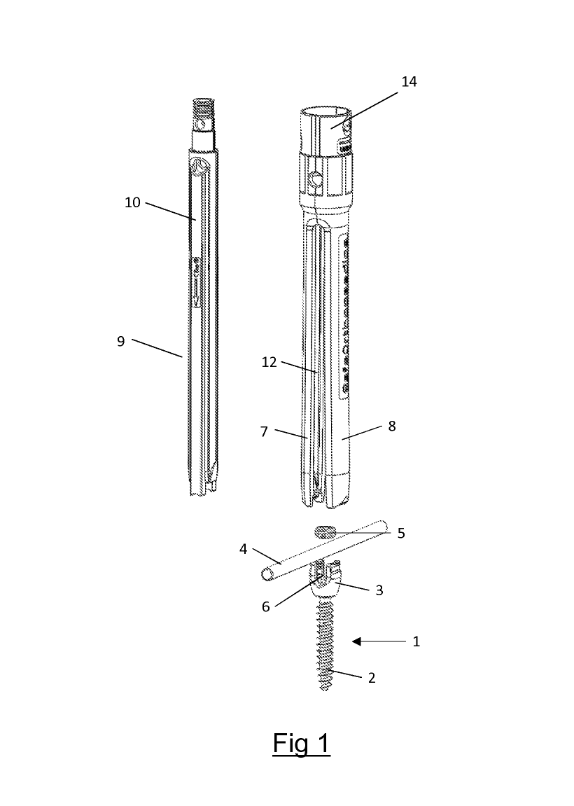

FIG. 1 is an overall view of the component elements of a surgical instrument of the invention.

### DETAILED DESCRIPTION

It comprises a pedicle screw (1) designed to be fastened to a vertebra, and comprising bone anchor means (2) extended by head (3) that is split for the purpose of receiving an intervertebral link rod (4). When the rod (4) is in place, a cap (5) that co-operates with the head (3) via a thread comes to lock the resulting assembly.

In a particular configuration, the head of the screw (3) is arranged to be U-shaped, the U-shape (6) being designed to receive the rod (4), with inside tapping designed to receive the cap (4) in order to secure together the screw (1) and the rod (4). The threaded portion (2) of the screw (1) may be mounted to be stationary or indeed to move relative to the head of the screw (3). This type of screw (1) having a cap (5) is in the public domain and is part of the prior art for spinal surgery for fastening vertebrae.

The material that is used most frequently for manufacturing such implants is titanium. In a particular configuration of the invention, the material used for the manufacturing may be any implantable material that is currently known or that is not yet known, such as polyether ether ketone (PEEK), stainless steel, cobalt-chrome, or indeed a composite based on glass fiber or carbon fiber. Coatings of the hydroxyapatite tricalcium phosphate (HATCP) type or of other types may also be applied to improve the bone anchoring or indeed the overall strength of the implant.

Fitting this screw (1) and putting the rod (4) in place, and then locking it with the cap (5) are achieved by an instrument described in Prior Art Patent PCTFR10/000880.

FIG. 1 shows an additional instrument, which is the main subject of the present patent application, and which differs from the prior art instrument mainly by the configuration of its proximal end (i.e. the end closer to the vertebrae and further from the surgeon).

In the non-limiting example described, it comprises two single-use legs (7, 8) and an insert (9). The insert (9) is entirely identical to the insert described in Prior Patent PCT/FR10/000880.

FIGS. 6 and 7 of Prior Patent PCT/FR10/000880 show that the tube carries a locking element (8) as pre-fitted to the cap (3), and how it is interconnected in the fitting tube (7) for fitting the screw (2).

The insert (9) is arranged to hold the cap (5) in secure manner in its proximal portion.

The cap (5) is held by tapping provided in the proximal end of the insert (9).

Alternatively, the cap (5) may be held by wedging or clipping.

The outside shape of the insert is provided with two longitudinal slideways (10, 11) that are of dovetail section and that are of shape complementary to the inside shape of the guide channels (12, 13) provided in the legs (7, 8).

These dovetails that are complementary between the two legs (7, 8) and the insert (9) strengthen the resulting assembly, thereby avoiding any risk of the two legs coming apart or being detached from each other under high stresses, and imparting higher bending and torsion strength to the assembly.

The advantage of such an insert is that the step of placing the cap (5) on the head (3) of the screw (1) and the step of locking the legs (7, 8) in position on the screw head are performed simultaneously. In the same way, the insert (9) makes it possible to put the rod (4) in place concomitantly with locking the two legs in position relative to each other.

The two legs (7, 8) are semi-cylindrical in cross-section. Their proximal portions are slightly divergent in order to facilitate force-fitting them onto the head (3) of the screw by moving them longitudinally.

The two legs (7, 8) are not parallel but rather they deviate slightly in such a manner as to avoid unstressed slippage of the insert in the proximal direction. The slight angle formed by the two legs provides braking that stabilizes the insert in the absence of longitudinal force exerted by the user. The legs (7, 8) are joined at their distal ends by an annular zone or by a handle (14).

The proximal ends are free and can thus move apart slightly by elastic deformation. Conversely, when the insert (9) is engaged and advanced in the proximal direction, it comes to bring the two legs together and couple them together mechanically. Thus, once in place in the longitudinal zone, the insert (9) keeps the positions of the legs (7, 8) constant relative to each other.

The same applies to the ring (18) of the variant embodiment that is described below.

The insert and the ring act identically. The surgeon has the possibility of performing the locking by acting on the ring and/or on the insert. For example, the surgeon may perform initial locking by moving the ring into the proximal position, and may then reinforce the locking by engaging the insert.

The legs (7, 8) and the insert (9) may be manufactured from any currently known materials, such as composites, polymers, ferrous metals, and non-ferrous metals (aluminum), so long as they correspond to the criteria of biocompatibility related to the application. Preferably, the material used is recyclable in order to satisfy the requirements of environment protection. Coatings may also be applied in order to satisfy biomcompatibility criteria, or indeed in order to improve strength or other mechanical characteristics.

The mode of sterilization that is chosen is compatible with the characteristics of said materials as in the state of the art. Such sterilization is preferably performed using gamma irradiation or a specific ethylene oxide (ETO) sterilization method.

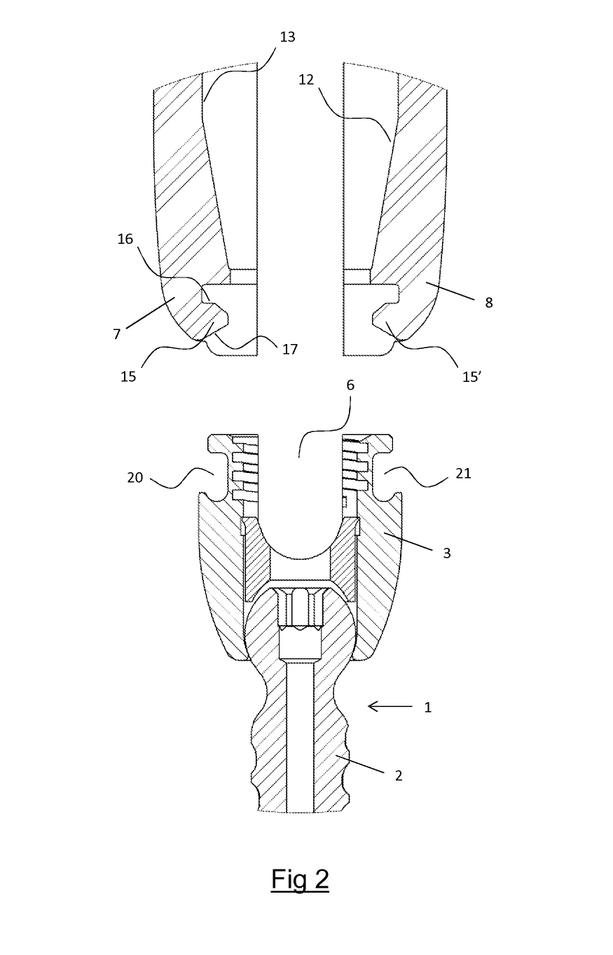

FIG. 2 is a detailed view of the proximal ends of the legs (7, 8).

It shows that each of the legs is provided with a respective lug (15, 15′) extending in a transverse direction. The height of the lugs is defined to enable them to be engaged laterally into the groove (20, 21) provided in the head of the screw.

Each lug has a plane distal face (16) that is slightly inclined at a slope of about 40° relative to the transverse plane. Said distal face (16) forms a shoulder suitable for co-operating with a proximal complementary shoulder of the head of the screw. Such co-operation makes it possible to anchor the instrument longitudinally to the head of the screw. It is thus possible to exert axial forces without detaching the instrument from the screw (1), e.g. so as to push the rod (4) into the U-shaped slot in the screw (1).

The lug (15) also has a proximal face (17) forming a bevel, with a slope of about 30 degrees relative to the transverse plane, so as to facilitate the engagement by clipping onto the head of the screw.

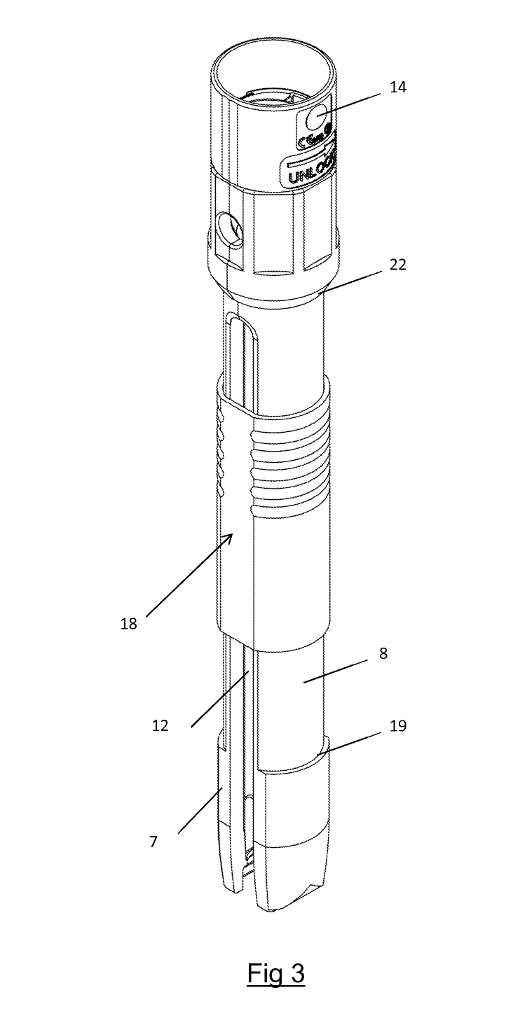

FIG. 3 shows a variant embodiment of an instrument, in which the legs (7, 8) are surrounded by a slidably mounted ring (18). The inside cross-section of the ring is complementary to the outside envelope of the legs (7, 8). It has two semi-circular arcs interconnected by straight segments if the legs are semi-cylindrical and separated by an opening for allowing the intervertebral rod to pass through.

For example, the height of the ring (18) is 50 millimeters long.

One of the legs (or both of them) has proximal (19) and distal (22) abutments for limiting the stroke of the ring (18). The proximal abutment is situated at about 30 millimeters from the proximal end. The distal abutment is constituted by the link element (14).

When the ring (18) is in the distal position, the legs can be moved apart to come to engage onto the head of the screw. When the ring (18) is pushed back into the proximal position, it brings the legs (7, 8) closer together onto the head of the screw, and locks the instrument on the head of the screw.

The surgical instrument of the invention can be part of a single-use instrument kit that is packaged in sterile packaging and that is designed for fitting or for removing implants.

The insert is then common to the basic instrument and to the additional instrument of the present patent.

The kit may further comprise a plurality of other instruments and, where applicable, implants making it possible to perform all of the surgical gestures required for fitting or for removing the implants.

When it is made of single-use sterile polymer materials, such an instrument kit offers many advantages such as the advantage of reducing the overall cost of spinal surgery, and of guaranteeing non-contamination from one patient to another, and thus of significantly reducing the number of hospital-acquired infections.

In non-limiting and in non-restrictive manner, the instrument kit makes it possible to perform the following surgical gestures: putting screws in place in the pedicles of vertebrae, bending the rod to match the anatomy of the patient, putting the rod in place regardless of the insertion forces required, putting the cap in place, performing instrumented vertebrae correction movements of the compression and distraction type, and performing controlled and safe final tightening of the cap.

1-11. (canceled)

12. A surgical instrument for fastening vertebrae via the posterior approach or via the posterolateral approach, the instrument comprising:
a tubular element formed of two legs separated by a recessed longitudinal zone, said legs being interconnected at their distal ends;
each of the legs having, in its inside surface, a guide channel of dovetail cross-section; and
an insert having two diametrically opposite longitudinal slideways of dovetail cross-section complementary to the cross-section of said guide channels; and
said instrument comprising the proximal end of each of the legs has anchor means suitable for coming to engage in complementary means by a transverse movement of said legs.

13. The surgical instrument for fastening vertebrae via the posterior approach or via the posterolateral approach according to claim 12, wherein the proximal end of each of the legs is provided with a transverse lug suitable for being engaged in a groove in the head of a pedicle screw, by lateral elastic deformation of the legs.

14. The surgical instrument according to claim 12, wherein said lug has a proximal surface that is substantially conical, for forming an insertion canal that opens out in the distal direction, said canal having, in the locked position, a shape complementary to the outside envelope of the head of the screw.

15. The surgical instrument according to claim 12, wherein, at rest, said legs diverge in the proximal direction.

16. The surgical instrument according to claim 12, wherein the legs are mounted to move between a position in which the proximal ends are spaced apart from each other beyond a rest position of said legs, and a position in which the proximal ends are brought closer together by the insert being engaged in the longitudinal zone.

17. The surgical instrument according to claim 12, wherein, said legs are of semi-cylindrical shape.

18. The surgical instrument according to claim 12, wherein the interconnection means for interconnecting the two legs are constituted by an annular distal portion extending said legs.

19. The surgical instrument according to claim 12, wherein said interconnection means for interconnecting the two legs are constituted by a handle that interconnects said legs.

20. The surgical instrument according to claim 12, wherein the two legs form separate parts, interconnected at their distal ends by interconnection means.

21. The surgical instrument according to claim 12, further comprising: a ring surrounding the two legs, said ring being mounted to move longitudinally between a proximal position in which it locks the legs on the head of the screw and a distal position in which it enables the legs to move transversely with a view to releasing the head of the screw.

22. The surgical instrument system for fastening vertebrae via the posterior approach or via the posterolateral approach, said system comprising a first instrument according to claim 12, and second instrument comprising a tubular element formed of two legs separated by a recessed longitudinal zone and joined at their distal ends by interconnection means, each of said legs having, in its inside surface, a guide groove of dovetail cross-section, the system further comprising said insert being common to the first instrument and to the second instrument.
